(19) United States
(12) Patent Application Publication
(10) Pub. No.: US 2023/0236136 A1
(43) Pub. Date: Jul. 27, 2023

(54) Dielectric Measurement of Construction Materials

(71) Applicant: Earth Science Systems, LLC, Wheat Ridge, CO (US)

(72) Inventor: Charles P. Oden, Wheat Ridge, CO (US)

(21) Appl. No.: 17/693,779

(22) Filed: Mar. 14, 2022

Related U.S. Application Data
(60) Provisional application No. 63/302,202, filed on Jan. 24, 2022.

(51) Int. Cl. G01N 22/00

(52) U.S. Cl. CPC G01N 22/00

(57) ABSTRACT

An apparatus and methods for measuring dielectric constant of prepared samples of construction materials or measuring dielectric constant of a surface of a structure fabricated from the construction materials is provided in the present invention. The present invention provides use of a dielectric waveguide to couple energy from an impulse radar to create the apparatus for testing building materials, where the dielectric waveguide is longer than one wavelength so that the Material Under Test (MUT) is in the far-field region of radar antennas.

### CROSS REFERENCE TO RELATED APPLICATIONS

This application claims priority on U.S. Provisional Patent Application No. 63/302,202, entitled “Dielectric Measurement of Construction Materials”, filed on Jan. 24, 2022, which is incorporated by reference herein in its entirety and for all purposes.

### FIELD OF THE INVENTION

The present invention relates to the field of dielectric measurement of construction materials, and more particularly, to an apparatus and method for measuring the dielectric properties of construction materials such as asphalts, concretes, cements, soils, sands, aggregates etc.

### BACKGROUND OF THE INVENTION

In general, the construction of pavements, roads, and buildings etc. require testing of construction materials for qualities purposes such as for compaction, density and moisture. Generally, there are two types of tests that are performed for checking the qualities of the construction materials, which are destructive tests and non-destructive tests.

The destructive tests are performed either in laboratory or in the field. In the laboratory destructive tests, samples are prepared, for example with an asphalt gyratory compactor, and various material properties are studied to determine the best mix design. In the field destructive tests, samples are cored from test strips or newly constructed roads or existing roads, new pavements or existing pavements. The material properties of these samples are then used to evaluate whether the test strip or newly constructed roads or existing roads, new pavements or existing pavements meets the design criteria and whether these are in good operating condition or in need of repairs.

Measuring the dielectric properties of construction materials is needed in order to provide reference values for non-destructive testing. There are some methods and apparatuses for measuring the dielectric properties of the construction materials available in the prior arts.

Ground penetrating radar (GPR) is a technology that can be used to quickly scan a surface at a construction site to map the dielectric constant of the surface and also locate subsurface objects (References: D. Daniels, 2004, Ground Penetrating Radar (2nd Edition), IEE, London, Page 752; Z Leng, 2011, Prediction of In-Situ Apshalt Mixture Density Using Ground Penetrating Radar: Theoretical Development and Field Verification, PhD Thesis, U. Illinois—Urbana, Page 144; C. Chen, K Rao, R., Lee, 2003, A New Ultrawide-Bandwidth Dielectric-Rod Antenna for Ground-Penetrating Radar Applications, IEEE Trans. Antennas and Propagation, v. 51,n. 3, Pages 371-377.)

Coaxial surface probes are commonly used to measure the properties of construction materials. When applied to construction materials, these probes oftentimes have the similar dimensions as the grains or inclusions in construction materials and their sample volume is oftentimes smaller or on the same order as the aggregate size used in construction materials. To obtain a representative measurement value, many measurements must be made and then averaged. Large surface probes can be fabricated, but this requires expensive and precise machining. Furthermore, these probes are typically operated using a vector network analyzer which are not typically designed for field use. A preferred solution employs ground penetrating radar (GPR) technologies because these technologies can be used to scan large areas on construction sites. (References: J. B. Salsman and S. P. Holderfield, 2011, A Technique for Measuring the Dielectric Properties of Minerals at Microwave Heating Frequencies Using an Open-Ended Coaxial Line, U.S. Dep. Of Interior, Bureau of Mines, RI9519, Page 19; B. Filali, F. Boone, J. Rhazi, G. Ballivy, 2008, Design and Calibration of a Large Open-Ended Coaxial Probe for the Measurement of the Dielectric Properties of Concrete, IEEE Trans. Microwave Theory and Techniques, v. 56, n. 10; and J. Senior, 2002, Optical Fiber Communications, PHI, 2nd Edition).

Electromagnetic waveguides can take many forms including a coaxial cable, a pair of parallel wires, and a long cylinder of dielectric material. The theory of cylindrical dielectric waveguides is described in these references (E. Snitzer, 1961, Cylindrical Dielectric Wave guide Modes, J. Opt. Soc. Am. 51, Page 491-498; and M Legenkiy and A. Butrym, 2011, Pulse Signals in Open Circular Dielectric Waveguide, Progress In Electromagnetics Research Letters, Vol. 22, Page 9-17).

Further, it is understood that prepared asphalt samples such as gyratory compaction pucks are routinely made during the asphalt mix design process and during large paving operations for process control. There are standard and routine laboratory methods for measuring the density and compaction of these prepared samples (Reference: Standard Test Method for Bulk Specific Gravity and Density of Non-Absorptive Compacted Asphalt Mixtures-ASTM D2726/D2726M-17), and by measuring both the density/compaction and the dielectric constant a mapping of dielectric constant to density/compaction can be made. This in turn allows dielectric measurements made of newly constructed asphalt to be converted onto compaction/density readings. These readings provide maps of compaction that project managers can use to insure that new construction has been completed according to the required specifications. Improper asphalt compaction is one of the largest problems that cause pavements or roads to deteriorate prematurely. Similarly, proper soil compaction is critical for providing adequate structural support for a wide variety of structures such as roads, buildings, dams, levees, etc.

U.S. patent Ser. No. 10/938,099 describes an apparatus and method for measuring the dielectric constant of a material, where the apparatus includes a ground penetrating radar (GPR) antenna measuring the surface dielectric of the material over a predefined area. The apparatus also comprises a dielectric spacer, disposed directly between the ground penetrating radar (GPR) antenna and the sample under test to facilitate energy coupling between the antenna and the sample under test. The thickness of the dielectric spacer is designed so that diffractions off the sides of the sample do not interfere with the reflected wave from the Material Under Test (MUT), and multiple reflections traveling through the dielectric spacer do not interfere with the reflected wave from the Material Under Test (MUT). The result is that the dielectric spacer is typically thinner than a wavelength and therefore the sample material is in the near-field region of the antennas in the impulse radar.

The center frequency of impulse radars used for investigating construction materials is typically 1-3 GHz. In this frequency band, the dielectric spacer used in U.S. patent Ser. No. 10/938,099 is less than one wavelength thick. This spacer thickness places the sample in the near-field antenna region where the sample can load the antenna elements and change the antenna response, whereas placing the samples in the far-field region does not change the antenna response. Additionally for prepared samples such as an asphalt puck from a gyratory compactor, if the samples are less than one wavelength from dipole transmitting antennas then the dielectric spacer method may not illuminate the sample material in a uniform manner

Hence, it is desirable to transmit waves through the entire specimen to obtain a measurement that represents the entire sample, not just a portion of the sample.

So in order to solve the above stated problems, the present invention provides an improved apparatus and method for measuring the dielectric properties of construction materials, where a long waveguide structure is used, so that the sample or Material Under Test (MUT) is placed in the far-field region of the radar's antennas. Furthermore, certain waveguide modes will provide a more uniform illumination of the sample under test.

### SUMMARY OF THE INVENTION

Aspects of the present invention provide use of a dielectric waveguide to couple energy from an impulse radar to create an apparatus for testing construction materials.

In one aspect of the present invention provides a dielectric waveguide that has a length of more than one wavelength.

One aspect of the present invention provides an apparatus and methods for measuring the dielectric constant of a prepared sample of asphalt, concrete, cement, soil, sand or aggregate. The apparatus for measuring dielectric constant of the prepared sample includes an impulse radar that is disposed on top of a dielectric waveguide, where the impulse radar contains a set of transmitting antennas (TX) and a set of receiving antennas (RX). The dielectric waveguide is used to focus the radar energy emitted by the transmitting antennas (TX) into the prepared sample of the Material Under Test (MUT). After the electromagnetic (EM) wave travels through the sample, a metallic reflector placed on the distal side of the sample reflects the radar energy back through the sample, through the waveguide, and to the receiving antennas (RX) in the impulse radar. By measuring the two-way travel time of the electromagnetic (EM) wave and the thickness of the sample, the electromagnetic (EM) wave velocity and dielectric constant for the sample material are determined.

The apparatus for measuring the dielectric constant of a fabricated sample of construction material or a fabricated surface of construction material (i.e., Material Under Test or MUT), the apparatus comprising an impulse radar assembly with integrated antennas, a dielectric waveguide, where the antennas include a set of transmitting antennas and a set of receiving antennas, where the set of transmitting antennas emit electromagnetic (EM) waveforms that travel through the dielectric waveguide to the Material Under Test (MUT), and reflect back from the Material Under Test (MUT) through the dielectric waveguide to the set of receiving antennas (RX) where the dielectric waveguide is configured for focusing the electromagnetic (EM) waveforms emitted by the transmitting antennas, where the length of the dielectric waveguide is more than one wavelength, and where the Material Under Test (MUT) is in a far-field region of the antennas.

In one aspect of the present invention the length of the waveguide is more than one wavelength, where the electromagnetic (EM) energy has a substantially uniform distribution in the waveguide, and the electromagnetic (EM) energy is concentrated inside the waveguide.

A method for measuring dielectric constant of a sample of Material Under Test (MUT) using the apparatus, the method comprising the following steps:

step 1, setting up the apparatus and recording electromagnetic (EM) waveforms,

step 2, adding a metal plate to a bottom of the waveguide and recording the electromagnetic (EM) waveforms then subtracting the waveforms recorded from the step 1,

step 3, setting up the apparatus on a sample and recording the waveforms,

step 4, adding another metal plate to a bottom of the sample and recording waveforms, and then subtracting the waveforms recorded from the step 3,

step 5, calculating a difference in arrival time between the waveforms from the step 4 and the step 2, that representing a two-way travel time for the electromagnetic (EM) waveforms travel through the sample,

step 6, measuring thickness of the sample, and

step 7, calculating the dielectric constant using a formula εᵣ=(ct/2 d)², where c is a speed of light, t is the two-way travel time, d is the thickness of the sample, and εᵣ is the dielectric constant.

A method for measuring dielectric constant of a surface of a Material Under Test (MUT using the apparatus, the method comprising the following steps:

step 1, setting up the apparatus, and recording electromagnetic (EM) waveforms,

step 2, setting up the apparatus on a surface and recording waveforms,

step 3, subtracting the waveforms recorded in step 2 from the step 1, where the resulting waveform is reflected wave amplitude for the surface, and

step 4, calculating the dielectric constant using a formula εᵣ=(Aᵢ+Aᵣ/Aᵢ−Aᵣ)², where Aᵣ is the reflected wave amplitude, A, is the incident wave amplitude and εᵣ is the dielectric constant.

The invention described herein is used to characterize how the dielectric constant of the material relates to composite material design parameters such as compaction or density.

In another aspect of the invention, the apparatus uses a dielectric waveguide to couple emissions from an impulse radar to the sample of the Material Under Test (MUT), or to the surface of a structure made from the material. The dielectric constant can be determined by measuring the travel time of an electromagnetic wave propagating through the prepared sample or from the amplitude of a wave that has reflected off of the surface of the material. The material sample typically is a width or diameter of at least 0.5 of the radar wavelength in the sample, and a thickness of 0.25 to 4 times the wavelength in the material sample at the radar's center frequency. Typical prepared samples are in the form of a cylinder, such as the asphalt pucks produced by a gyratory compactor which are typically 150 mm in diameter and 100 mm tall. Alternatively the sample could be a core taken from a roadway or a soil sample produced by a Proctor compaction mold.

In another aspect of the present invention provides that the length of the dielectric waveguide is more than one wavelength so that the Material Under Test (MUT) is in the far-field region of radar antennas. This configuration avoids the changing antenna response due to foreign objects within the near-field region. Furthermore, prepared samples are illuminated more uniformly so that more representative measurements of the sample can be made.

The invention will be used in the road construction industry where subgrade soils must be properly compacted, and for asphalt paving where the asphalt must be properly compacted.

The invention will also be used during the construction of other structures such as buildings, dams, embankments, etc. where the compaction/density of soils must be properly controlled.

### BRIEF DESCRIPTION OF THE DRAWINGS

The object of the invention may be understood in more details, and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1a illustrates an apparatus for measuring dielectric constant of a prepared sample of a Material Under Test (MUT), according to an embodiment of the present invention;

FIG. 1b illustrates the apparatus for measuring dielectric constant of a surface of a structure constructed from the Material Under Test (MUT), according to an embodiment of the present invention;

FIG. 2 illustrates a graph plotting the maximum allowable spacer thickness in time window for reception of clean reflected signals from test material using a dielectric spacer in the prior art;

FIG. 3 illustrates ray paths in a dielectric waveguide, according to an embodiment of the present invention;

FIG. 4 illustrates electric field polarizations for the dielectric waveguide, according to an embodiment of the present invention;

FIG. 5 illustrates a block diagram of a method measuring the dielectric constant of a sample, according to an embodiment of the present invention;

FIG. 6 shows the reference waveform along with the travel time response for two different prepared samples, according to an embodiment of the present invention;

FIG. 7 illustrates a block diagram of a method measuring the dielectric constant of a surface, according to an embodiment of the present invention; and

FIG. 8 shows reflected waveforms obtained from surfaces constructed from three different materials having different dielectric constant.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

As described herein with several embodiments, the present invention provides an apparatus and methods for measuring the dielectric constant of construction materials or measuring the dielectric constant of a surface of a structure fabricated/constructed from the material. The invention described herein is used to characterize how the dielectric constant of the material relates to composite material design parameters such as compaction or density.

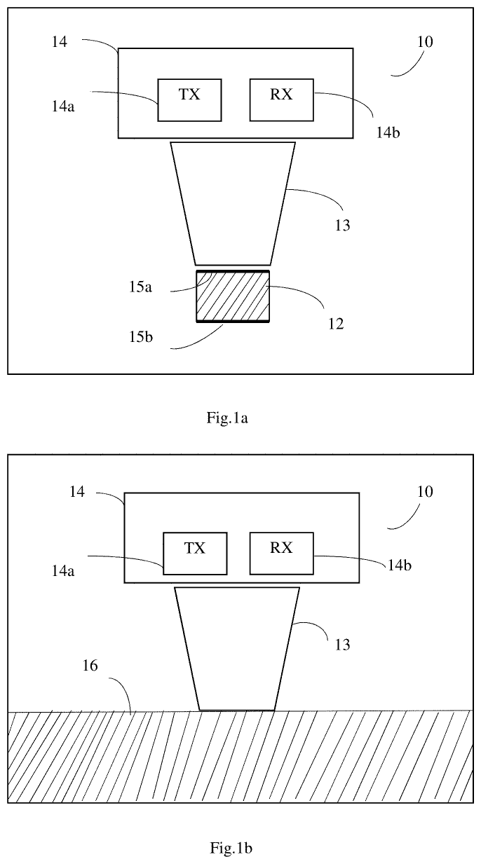

Now, the invention will be described in details herewith referring to the Figures. As shown in FIG. 1a, in one embodiment, the invention provides an apparatus 10 for measuring dielectric constant of a prepared sample 12 of a Material Under Test (MUT). As it can be seen in FIG. 1a, an impulse radar (a radar scanner) 14 is disposed on top of a dielectric waveguide 13, where the impulse radar 13 contains with a set of transmitting antennas (TX) 14a and a set of receiving antennas (RX) 14b. The dielectric waveguide 13 is used to focus the electromagnetic (EM) waves (radar energy) emitted by the transmitting antennas (TX) 14a into the prepared sample 12 of the Material Under Test (MUT). After the electromagnetic (EM) waves travel through the sample 12, a metal plate (metallic reflector) 15a placed on the distal side of the sample 12 reflects the electromagnetic (EM) waves (radar energy) back through the sample 12, through the waveguide 13, and to the receiving antennas (RX) 14b in the impulse radar 14. By measuring the two-way travel time of the electromagnetic (EM) wave and the thickness of the sample 12, the electromagnetic (EM) wave velocity and dielectric constant for the sample material 12 are determined.

Accordingly as shown in FIG. 1b, in another embodiment, the invention provides an apparatus 10 for measuring dielectric constant of a surface 16 of a structure constructed from the Material Under Test (MUT). As it can be seen in FIG. 1b, the impulse radar 14 and dielectric waveguide 13 assembly can be placed directly on the surface 16 that has been formed from a construction material such as asphalt, concrete, cement, soil, sand or aggregate. The dielectric constant of the construction material can be measured observing the amplitude of the reflected wave. An electromagnetic (EM) wave is transmitted by the impulse radar 14 though the dielectric waveguide 13, which reflects off of the surface 16 of the construction material and travels back through the dielectric waveguide 13 to the impulse radar 14.

In the embodiments of the invention, the apparatus 10 uses a dielectric waveguide 13 to couple emissions from an impulse radar 14 to the sample 12 of the Material Under Test (MUT), or to the surface 16 of a structure made from the material. The dielectric constant can be determined by measuring the travel time of the electromagnetic (EM) wave propagating through the prepared sample 12, or from the amplitude of a wave that has reflected off of the surface 16 of the material. The material sample typically is a width or diameter of at least 0.5 of the radar wavelength in the sample 12, and a thickness of 0.25 to 4 times the wavelength in the sample 12 at the radar's center frequency. Typical prepared samples 12 are in the form of a cylinder, such as the asphalt pucks produced by a gyratory compactor which are typically 150 mm in diameter and 100 mm tall. Alternatively the sample 12 could be a core taken from a roadway or a soil sample produced by a Proctor compaction mold.

The use of the dielectric waveguide 13 to couple energy from the impulse scanner 14 to the Material Under Test (MUT) has not been used previously to create an apparatus 10 for testing building materials using the impulse radar 14.

In the embodiments, the dielectric waveguide 13 is longer than one wavelength. So, the Material Under Test (MUT) is placed in the far-field region of the antennas 14a and 14b of the impulse radar 14. Compensation routines are employed to account for the dielectric waveguide 13 response that may include reflections off of the interior side of the dielectric waveguide 13.

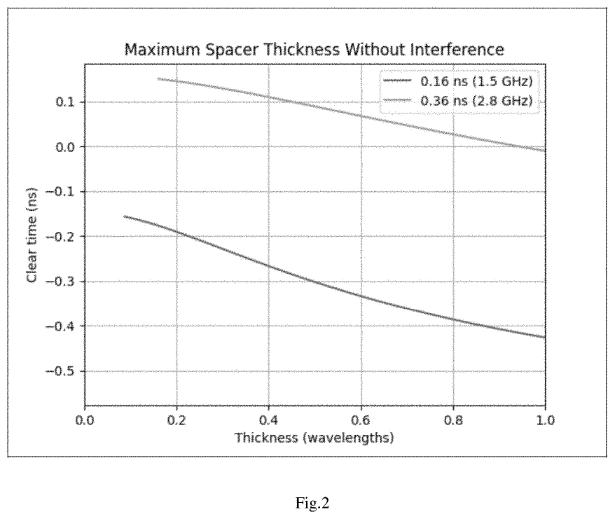

As discussed above, the dielectric waveguide 13 is significantly longer than the constraints put for the dielectric spacer of the prior art U.S. patent Ser. No. 10/938,099. The spacer is constrained to be sufficiently thin that the waves reflecting off the edges of the sample do not interfere with earliest reflected waves from the center of the sample. The antenna offset of 6 cm, impulse width of 0.36 ns, and a spacer dielectric constant of 3 are shown in FIG. 2. In the FIG. 2, the graph plots the maximum allowable spacer thickness. The center frequency of impulse radars used for investigating construction materials is typically 1-3 GHz, and whereas FIG. 2 shows the spacer thickness calculated based on specification details of the U.S. patent Ser. No. 10/938,099. The specified spacer thickness places the Material Under Test (MUT) within the near-field region of the radar antennas. No lateral constraints on the spacer are mentioned, and internal reflections off of the sides of the spacer are not addressed.

In contrast, the present invention provides that the length of the dielectric waveguide 13 is more than one wavelength so that the Material Under Test (MUT) is in the far-field region of antennas 14a and 14b of the impulse radar 14. This configuration avoids the changing antenna 14a and 14b response due to foreign objects within the near-field region. Furthermore, prepared samples are illuminated more uniformly so that more representative measurements of the sample can be made.

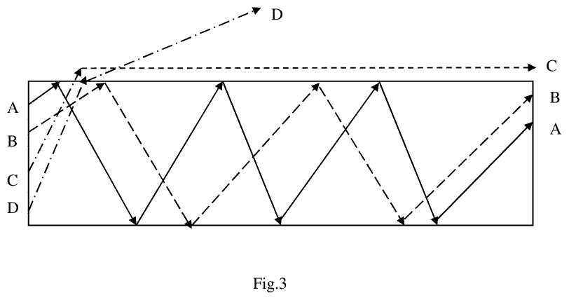

The dielectric waveguide 13 is long narrow structure composed of a dielectric inner core material that is surrounded by material with a lower dielectric value. This structure preferentially guides energy along its length. Since plastics have a higher dielectric constant than air, a long plastic prism or cylinder will function as the dielectric waveguide 13. FIG. 3 illustrates ray paths in the dielectric waveguide 13, where electromagnetic (EM) waves (energy) traveling in the structure with ray paths less than the critical angle will be completely internally reflected and travel inside the dielectric waveguide 13, which are represented as ray paths A and B. The electromagnetic (EM) Wave (energy) traveling at the critical angle will couple to the outside of the cylinder and travel along the structure at the speed of light (c), which is represented as a ray path C. Lastly, waves (energy) with ray paths greater than the critical angle will move away from the structure and these components will attenuate rapidly with distance along the dielectric waveguide 13, which is represented as a ray path D.

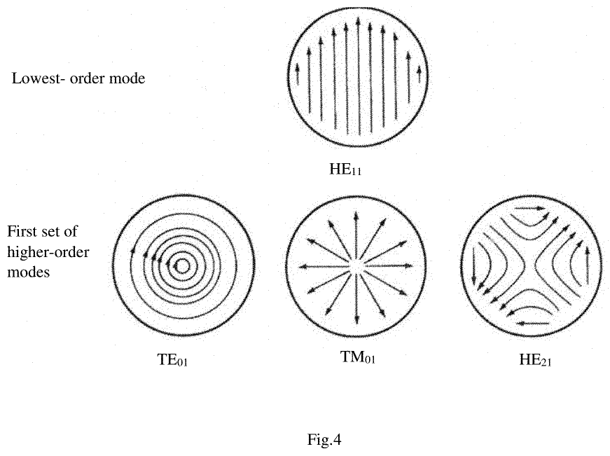

FIG. 4 shows electric field polarization for the dielectric waveguide 13, In general, the mode structures supported by the dielectric waveguide 13 can be complicated with TE, TM, and hybrid modes HE and EH. The response of a dielectric waveguide 13 is characterized by its normalized frequency (V). For frequency (V) below 2.405, the dielectric waveguide 13 only supports a single mode (HE11, sometimes called the dipole mode), and for large frequency (V) values, the number of supported modes is approximately V²/2. Accordingly, a plastic waveguide with dimensions listed previously will support about 8 modes.

However, only the lowest order mode (HE11) is linearly polarized and since the radar antennas are linearly polarized, most of the waveguide energy will be in the lowest order mode and higher order modes are only weakly excited (Senior, 2002).

In one embodiment, the waveguide 13 dimensions are selected to couple to the impulse radar 14 on one side and to a gyratory compactor puck on the other side. In the USA, a standard gyratory puck is 150 mm in diameter and 100 mm thick. In order to couple between a 2 GHz Ground penetrating radar (GPR) scanner and a gyratory puck, a tapered dielectric waveguide 13 with a diameters of 150 mm (1.6λ) and 210 mm (2.3λ), and a length of 240 mm (2.7λ) was fabricated. Despite the slow taper, the waveguide 13 phenomenon described in FIG. 3 still apply, although the changing diameter could change the mode structure supported by the waveguide 13. But since only the HE11 mode is substantially excited, this does not adversely affect the waveguide 13 performance

By using the waveguide 13 with diameters greater than a wavelength, most of the energy is concentrated inside the waveguide 13 and only a small amount travels outside the waveguide 13. The external wave (ray path C) in FIG. 3 that travels along the outside of the waveguide 13 can only be exited with a ray path at one specific angle, and since the radar impulse 14 produce a broad distribution of ray paths, the energy in this ray path C is substantially less than that of internally the guided waves. Finally, when using a waveguide 13 longer than one wavelength, the changing properties of the Material Under Test (MUT) do not affect the response of the antennas 14a and 14b on the other side of the waveguide 13.

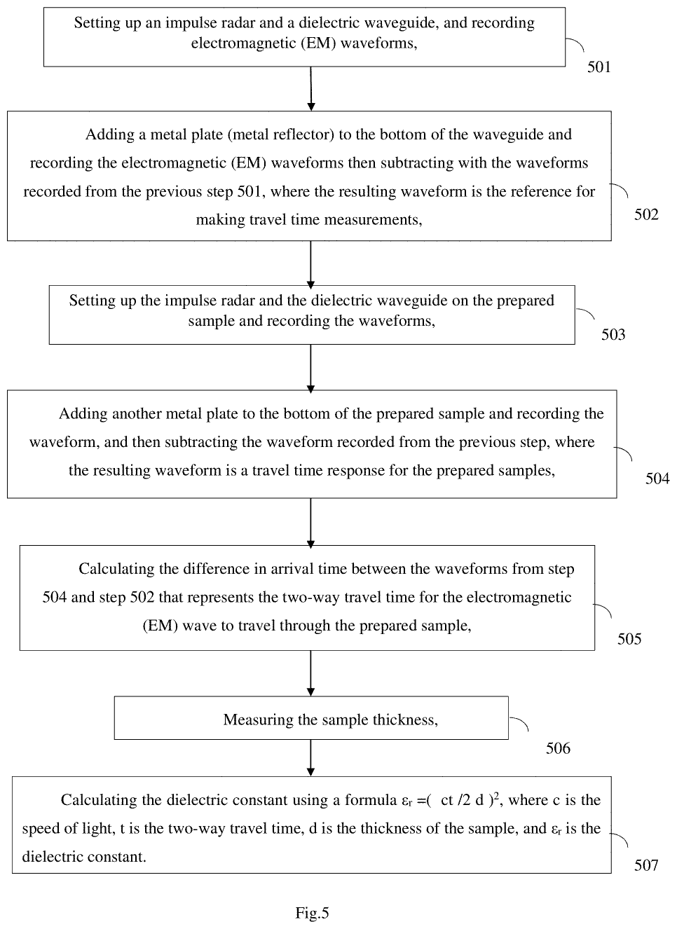

In some embodiments, as shown in FIG. 5 the dielectric constant of a prepared sample 12 can be measured using the following procedure:

Step 501: Setup the impulse radar 14 and the dielectric waveguide 13 so that there are no other objects in the immediate vicinity of the waveguide 13. Record the background electromagnetic (EM) waveform for this setup.

Step 502: Add a metal plate 15a to the bottom of the waveguide 13 and record the electromagnetic (EM) waveforms. Subtract the background waveform recorded from the previous step 501. The resulting waveform is the reference for making travel time measurements.

Step 503: Setup the impulse radar 14, the dielectric waveguide 13, and the prepared sample 12 as shown in the FIG. 1a. Insure that there are no other objects in the vicinity of the waveguide 13 or sample 12. Record the radar background waveform to this setup.

Step 504: Add a metal plate 15b to the bottom of the prepared sample 12 and record the waveform. Subtract the background waveform recorded from the previous step 503. The resulting waveform is the travel time response for the prepared samples 12.

Step 505: The difference in arrival time between the waveforms from step 504 and step 502 represents the two-way travel time for the electromagnetic wave to travel through the prepared sample 12.

Step 506: Measure the sample thickness (e.g., with a caliper).

Step 507: After measuring the sample thickness, the dielectric constant can be calculated with this formula:

εᵣ=(ct/2d)², where c is the speed of light, t is the two-way travel time, d is the thickness of the sample, and εᵣ is the dielectric constant.

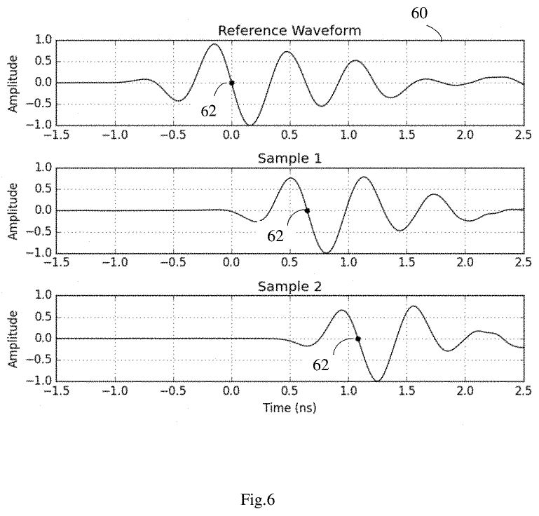

FIG. 6 shows the reference waveform 60 along with the travel time response for two different prepared samples 12 (sample 1 and sample 2). The dots 62 on the plot indicate the arrival times used to calculate the two-way travel time. The use of the HE11 mode excites nearly the entire cross section of the waveguide 13 which helps to more fully and uniformly illuminate the prepared sample 12.

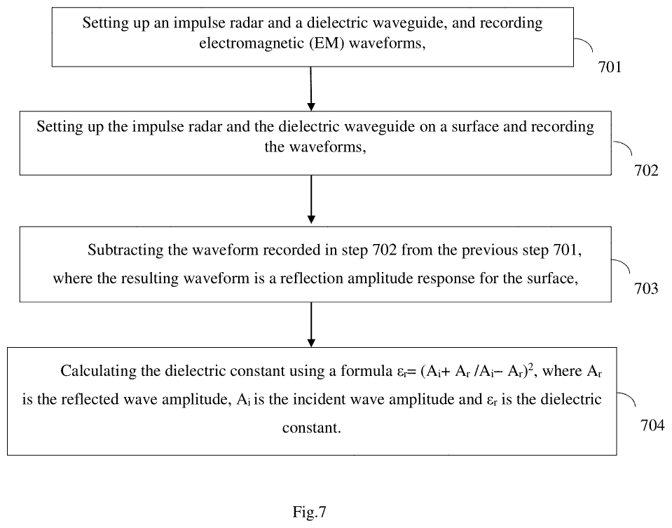

As shown in FIG. 7, the dielectric constant of a surface 16 constructed using construction materials such as asphalt, concrete, cement, soil, sand or aggregate can be measured using the test configuration as shown FIG. 1b. The procedure is outlined below:

Step 701: Setup the impulse radar 14 and the dielectric waveguide 13 so that there are no other objects in the immediate vicinity of waveguide 13. Record the electromagnetic (EM) waveforms for this setup. This is the background fixture response that will be subtracted from other measurements.

Step 702: Place the impulse radar 14 and the dielectric waveguide 13 on the surface 16 and record the electromagnetic (EM) waveforms.

Step 703: Subtract the waveform recorded from the previous step. The resulting waveform is the reflection amplitude response for the surface 16.

Step 704: After measuring the reflection amplitude, the dielectric constant can be calculated with this formula:

εᵣ=(Aᵢ+Aᵣ/Aᵢ−Aᵣ)², where Aᵢ is the reflected wave amplitude, Aᵣ is the incident wave amplitude and εᵣ is the dielectric constant.

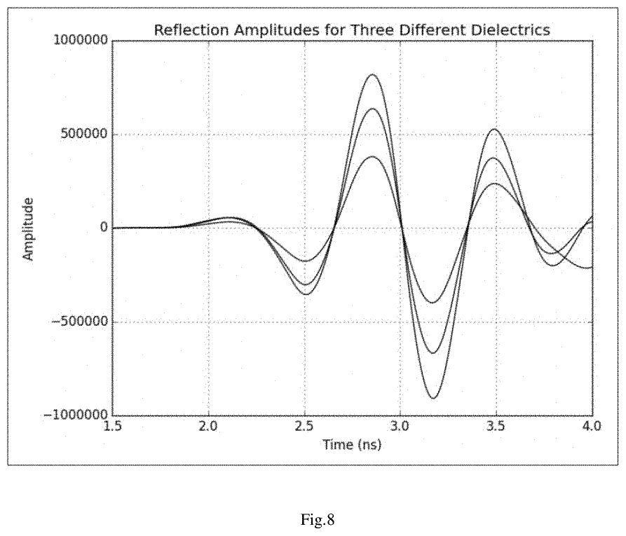

FIG. 8 shows reflected waveforms obtained from surfaces 16 constructed from three different dielectric materials using the test configuration shown in the FIG. 1b.

To examine the surface area 16 sensed by the setup shown in the FIG. 1b, experiments were conducted using a 2 GHz impulse radar 14, a tapered dielectric waveguide 13 with a diameters of 150 mm (1.6λ), 210 mm (2.3λ), and a length of 240 mm (2.7λ), and several different sized metal disk reflectors. Table 1 below shows that most of the response comes from the area directly below the bottom of the waveguide 13. This is due to the fact that with waveguide 13 diameters greater than a wavelength, most of the energy is concentrated inside the waveguide 13 and only a small amount travels outside the waveguide 13.

TABLE 1
Surface reflection response as a function of spot
diameter on the surface beneath the waveguide.

| Diameter | % of total response |
|---|---|
| 150 mm | 92% |
| 300 mm | 95% |
| 450 mm or larger | 100% |

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

What is claimed is:

1. An apparatus for measuring dielectric constant of construction materials of a sample or a fabricated surface by the construction materials of a Material Under Test (MUT), the apparatus comprising:
an impulse radar assembly comprising antennas;
a dielectric waveguide,
wherein,
the antennas including a set of transmitting antennas and a set of receiving antennas, where the set of transmitting antennas emit electromagnetic (EM) waves (radar energy) into the dielectric waveguide to couples the energy to the Material Under Test (MUT), which reflects back from the Material Under Test (MUT) through the dielectric waveguide to the set of receiving antennas (RX);
where the dielectric waveguide is configured for focusing the electromagnetic (EM) energy emitted by the transmitting antennas, where a length of the dielectric waveguide is more than one wavelength, and where the Material Under Test (MUT) is in a far-field region of the antennas.

2. The apparatus of claim 1, wherein the length of the waveguide is more than one wavelength, where the electromagnetic (EM) energy has a substantially uniform distribution in the waveguide, and the electromagnetic (EM) energy is concentrated inside the waveguide.

3. The apparatus of claim 1, wherein the dielectric constant are determined by measuring a two way time travel of the electromagnetic (EM) waves penetrating through the construction materials and reflecting back from the construction materials.

4. The apparatus of claim 1, wherein the dielectric constant of the construction materials is measured observing an amplitude of the reflected waveforms.

5. The apparatus of claim 1, wherein the construction materials are including but not limited to asphalts, concretes, cements, soils, sand and aggregates.

6. The apparatus of claim 1, wherein a combination of the impulse radar assembly and the dielectric waveguide is placed directly on the surface fabricated from the construction materials.

7. The apparatus of claim 1, where the dielectric waveguide is long narrow structure composed of a dielectric inner core material surrounded by a material with a lower dielectric value.

8. A method for measuring dielectric constant of a sample of a Material Under Test (MUT) using an apparatus comprising of an impulse radar assembly including antennas, and a dielectric waveguide, where the antennas including a set of transmitting antennas and a set of receiving antennas, where a length of the dielectric waveguide is more than one wavelength, the Material Under Test (MUT) is in a far-field region of the antennas, the method comprising the following steps:
step 1, setting up the apparatus and recording electromagnetic (EM) waveforms,
step 2, adding a metal plate to a bottom of the waveguide and recording the electromagnetic (EM) waveforms then subtracting with the waveforms recorded from the step 1,
step 3, setting up the apparatus on a sample and recording the waveforms,
step 4, adding another metal plate to a bottom of the sample and recording waveforms, and then subtracting the waveforms recorded from the step 3,
step 5, calculating a difference in arrival time between the waveforms from step 4 and step 2, that representing a two-way travel time for the electromagnetic (EM) waves to travel through the sample,
step 6, measuring a thickness of the sample, and
step 7, calculating the dielectric constant using a formula εᵣ=(ct/2 d)², where c is a speed of light, t is the two-way travel time, d is the thickness of the sample, and εᵣ is the dielectric constant.

9. The method of claim 8, wherein the length of the waveguide is more than one wavelength, where the electromagnetic (EM) energy has a substantially uniform distribution in the waveguide, and the electromagnetic (EM) energy is concentrated inside the waveguide.

10. The method of claim 8, wherein the dielectric constant is determined by measuring a two way time travel of the electromagnetic (EM) waves that penetrate through the construction materials and reflecting back from the construction materials.

11. The method of claim 8, wherein the construction materials are including but not limited to asphalts, concretes, cements, soils, sand and aggregates.

12. The method of claim 8, where the dielectric waveguide is long narrow structure composed of a dielectric inner core material surrounded by a material with a lower dielectric value.

13. A method for measuring dielectric constant of a surface of a Material Under Test (MUT) using an apparatus comprising of an impulse radar assembly including antennas, and a dielectric waveguide, where the antennas including a set of transmitting antennas and a set of receiving antennas, where the length of the dielectric waveguide is more than one wavelength, and the material under test (MUT) is in a far-field region of the antennas, the method comprising the following steps:
step 1, setting up the apparatus, and recording electromagnetic (EM) waveforms,
step 2, setting up the apparatus on the surface and recording waveforms,
step 3, subtracting the waveforms recorded in step 2 from the step 1, where the resulting waveform is reflected wave amplitude for the surface, and
step 3, calculating the dielectric constant using a formula εᵣ=(Aᵢ+Aᵣ/Aᵢ−Aᵣ)², where Aᵣ is the reflected wave amplitude, A, is the incident wave amplitude and εᵣ is the dielectric constant.

14. The method of claim 13, wherein the length of the waveguide is more than one wavelength, where the electromagnetic (EM) energy has a substantially uniform distribution in the waveguide, and the electromagnetic (EM) energy is concentrated inside the waveguide.

15. The method of claim 13, wherein the construction materials are including but not limited to asphalts, concretes, cements, soils, sand and aggregates.

16. The method of claim 13, wherein the dielectric constant of the construction materials is measured observing the amplitude of the reflected waveforms.

17. The method of claim 13, wherein a combination of the impulse radar assembly and the dielectric waveguide is placed directly on the surface fabricated from the construction materials.

18. The method of claim 13, where the dielectric waveguide is long narrow structure composed of a dielectric inner core material surrounded by a material with a lower dielectric value.
